(19) United States
(12) Patent Application Publication
(10) Pub. No.: US 2018/0094926 A1
(43) Pub. Date: Apr. 5, 2018

(54) Penetrometer

(71) Applicant: Fugro Engineers B.V., Nootdorp (NL)

(72) Inventors: Peter Nicolaas Looijen, Nootdorp (NL); David Steven Ruijtenbeek, Nootdorp (NL)

(21) Appl. No.: 15/724,414

(22) Filed: Oct. 4, 2017

(30) Foreign Application Priority Data
Oct. 4, 2016 (NL) 2017574

(51) Int. Cl. G01C 13/00; G01N 33/24; G01N 3/40

(52) U.S. Cl. CPC G01C 13/008; G01V 9/00; G01N 3/40; G01N 33/24

(57) ABSTRACT

A penetrometer arranged for detecting a transition zone from water to sediment of a seabed, comprising a cone with a cone sleeve and a cone tip and a force sensitive element or elements, wherein the force sensitive element or elements comprise a piezo-electric sensor which is provided in contact with the cone tip so as to respond to a force or forces applied to the cone tip. The piezo-electric sensor is supported by a rim inside the cone sleeve.

### CROSS-REFERENCE TO RELATED APPLICATIONS

This application claims priority to Netherlands Patent Application No. 2017574, filed on Oct. 4, 2016, and the specification and claims thereof are incorporated herein by reference.

### STATEMENT REGARDING FEDERALLY SPONSORED RESEARCH OR DEVELOPMENT

Not Applicable.

### THE NAMES OF PARTIES TO A JOINT RESEARCH AGREEMENT

Not Applicable.

### INCORPORATION BY REFERENCE OF MATERIAL SUBMITTED ON A COMPACT DISC

Not Applicable.

### STATEMENT REGARDING PRIOR DISCLOSURES BY THE INVENTOR OR A JOINT INVENTOR

Not Applicable.

### COPYRIGHTED MATERIAL

Not Applicable.

### BACKGROUND OF THE INVENTION

### Field of the Invention (Technical Field)

The present invention relates to a penetrometer arranged for detecting a transition zone from water to sediment of a seabed, comprising a cone with a cone sleeve and a cone tip and a force sensitive element or elements.

### Description of Related Art Including Information Disclosed Under 37 C.F.R. § § 1.97 and 1.98

Penetrometers are used in practice for detecting the exact position of the transition from water to the sediment of the seabed. This level forms a reference point for a subsequent soil measurement below the sea bottom, wherein it is a necessity to exactly know the level of penetration of the penetrometer with reference to the level of the seabed.

The known penetrometer used by industry is equipped with sensors embodied as strain gauges that are used for measuring a force reaction of the soil. In practice, however, this measurement with the known penetrometer equipped with strain gauges does not suffice because the top layer of the seabed is too soft to accurately measure the level of the seabed.

It is an object of the invention to provide a solution for this problem.

From GB-A-2466773 a penetrometer is known, comprising a cone with a cone sleeve and a cone tip and a force sensitive element or elements that may comprise piezo-electric elements, which is or are provided in contact with the cone tip so as to respond to a force or forces applied to the cone tip.

### BRIEF SUMMARY OF THE INVENTION

The penetrometer of the invention has the features of one or more of the appended claims.

Further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 shows a first embodiment of the penetrometer according to the invention; and

FIG. 2 shows a second embodiment of the penetrometer according to the invention.

### DETAILED DESCRIPTION OF THE INVENTION

In a first aspect of the invention the piezo-electric element is a piezo-electric sensor that is supported by a rim inside the cone sleeve. The force applied to the piezo-electric sensor is converted in an electrical signal which indicates when the penetrometer of the invention has reached the level of the seabed. Supported by the rim the piezo-electric sensor can thus be subjected to the force of the cone tip without being moved out of position to provide that the measurement of the seabed level is reliable and accurate. Accordingly, the penetrometer can be effectively used for detecting a transition from water to sediment of a seabed.

Beneficially the cone tip is provided with a shoulder arranged to cooperate with a frontal end of the cone sleeve for limiting a movement range of the cone tip in a direction towards the piezo-electric sensor. In this way damage to the piezo-electric sensor due to excessive loads by the cone tip is prevented.

Advantageously the cone sleeve is comprised within an outer sleeve, wherein the cone sleeve and the outer sleeve are longitudinally movable with respect to each other. By this arrangement it is possible that between the cone sleeve and the outer sleeve one or more high load force sensors, preferably strain gauges, are applied. The high load force sensors then can provide measurement signals of the force applied to the cone tip in a force measurement range for which the piezo-electric sensor is not suitable and would be prone to damage. The high load force sensors, preferably strain gauges, can in particular be used for force measurement when the shoulder of the cone tip rests on the frontal end of the cone sleeve.

The invention will hereinafter be further elucidated with reference to the drawing of an exemplary embodiment of an apparatus according to the invention that is not limiting as to the appended claims.

Whenever in the figures the same reference numerals are applied, these numerals refer to the same parts.

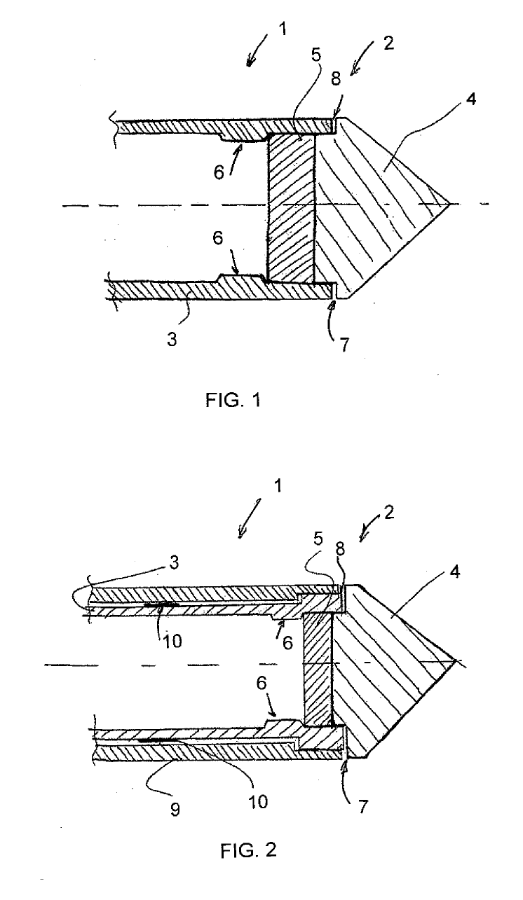

With reference to both FIG. 1 and FIG. 2, a first embodiment and a second embodiment of the penetrometer 1 of the invention is shown.

Both embodiments of FIG. 1 and FIG. 2 are arranged for detecting a seabed force and comprise a cone 2 with a cone sleeve 3 and a cone tip 4 and a force sensitive element or elements. The force sensitive element or elements comprise according to the invention at least a piezo-electric sensor 5 which is provided in contact with the cone tip 4 so as to respond to a force or forces applied to the cone tip 4.

The piezo-electric sensor 5 is in both embodiments of FIG. 1 and FIG. 2 supported by a rim 6 inside the cone sleeve 3.

Further the cone tip 4 is in both embodiments of FIG. 1 and FIG. 2 provided with a shoulder 7 arranged to cooperate with a frontal end 8 of the cone sleeve 3 for limiting a movement range of the cone tip 4 in a direction towards the piezo-electric sensor 5.

The embodiment shown in FIG. 2 differentiates from the embodiment shown in FIG. 1 in that the cone sleeve 3 is comprised within an outer sleeve 9, wherein the cone sleeve 3 and the outer sleeve 9 are longitudinally movable with respect to each other.

This makes possible that between the cone sleeve 3 and the outer sleeve 9 one or more strain gauges 10 or other high load force sensors are applied.

Although the invention has been discussed in the foregoing with reference to exemplary embodiments of the penetrometer of the invention, the invention is not restricted to these particular embodiments which can be varied in many ways without departing from the invention. The discussed exemplary embodiments shall therefore not be used to construe the appended claims strictly in accordance therewith. On the contrary the embodiments are merely intended to explain the wording of the appended claims without intent to limit the claims to these exemplary embodiments. The scope of protection of the invention shall therefore be construed in accordance with the appended claims only, wherein a possible ambiguity in the wording of the claims shall be resolved using these exemplary embodiments.

What is claimed is:

1. A penetrometer arranged for detecting a transition from water to sediment of a seabed, comprising a cone with a cone sleeve and a cone tip and a force sensitive element or elements, wherein the force sensitive element or elements comprise a piezo-electric element that is provided in contact with the cone tip so as to respond to a force or forces applied to the cone tip, wherein the piezo-electric element is a piezo-electric sensor that is supported by a rim inside the cone sleeve.

2. The penetrometer according to claim 1, wherein the cone tip is provided with a shoulder arranged to cooperate with a frontal end of the cone sleeve for limiting a movement range of the cone tip in a direction towards the piezo-electric sensor.

3. The penetrometer according to claim 2, wherein the cone sleeve is comprised within an outer sleeve, wherein the cone sleeve and the outer sleeve are longitudinally movable with respect to each other.

4. The penetrometer according to claim 3, wherein between the cone sleeve and the outer sleeve are one or more high load force sensors.

5. The penetrometer according to claim 4, wherein between the cone sleeve and the outer sleeve are one or more strain gauges.

6. The penetrometer according to claim 1, wherein the cone sleeve is comprised within an outer sleeve, wherein the cone sleeve and the outer sleeve are longitudinally movable with respect to each other.

7. The penetrometer according to claim 6, wherein between the cone sleeve and the outer sleeve are one or more high load force sensors.

8. The penetrometer according to claim 7, wherein between the cone sleeve and the outer sleeve are one or more strain gauges.

9. A method of using a penetrometer for detecting a sediment layer, wherein the penetrometer comprises a cone with a cone sleeve and a cone tip and a force sensitive element or elements, wherein the force sensitive element or elements comprise a piezo-electric sensor that is provided in contact with the cone tip so as to respond to a force or forces applied to the cone tip, wherein the penetrometer is used for detecting a transition from water to sediment of a seabed.
